(19) United States
(12) Patent Application Publication
(10) Pub. No.: US 2022/0403118 A1
(43) Pub. Date: Dec. 22, 2022

(54) WATER-BASED HYDROGEL, AND REACTION PRODUCT OF ISOSORBIDE EPOXIDE AND AMINES

(71) Applicant: ROQUETTE FRERES, Lestrem (FR)

(72) Inventors: René SAINT-LOUP, LOMME (FR); Audrey SAHUT, LESTREM (FR)

(21) Appl. No.: 17/756,396

(22) PCT Filed: Nov. 26, 2020
(86) PCT No.: PCT/FR2020/052182

§ 371 Date: May 24, 2022

(30) Foreign Application Priority Data
Nov. 27, 2019 (FR) FR1913342

(51) Int. Cl. C08J 3/075; C08G 59/50; C08G 59/26; A61K 31/77; A61K 9/06; A61K 8/04; A61K 8/86

(52) U.S. Cl. CPC C08J 3/075; C08G 59/504; C08G 59/26; A61K 31/77; A61K 9/06; A61K 8/042; A61K 8/86; C08J 2363/00; A61K 2800/10

(57) ABSTRACT

The invention relates to a hydrogel based on water, on hydrosoluble monomeric or polymeric isosorbide epoxide and on hydrosoluble amine, to the method for preparing same and to the use thereof.

The present invention relates to a hydrogel and more particularly to a hydrogel based on water, and on the reaction product of hydrosoluble monomeric or polymeric isosorbide epoxide and of hydrosoluble amine, to the method for preparing same and to the use thereof.

### INTRODUCTION

Hydrogels are a class of products consisting of a reversible physical network or irreversible chemical network in which water can be trapped. These hydrogels are insoluble in water. Hydrogels are highly absorbent polymeric materials and are used in various applications.

There are several methods to prepare irreversible chemical hydrogels, for example crosslinking water-soluble polymers or swelling dry hydrophilic polymer networks in water.

The majority of polymeric hydrogels in the literature contain polyester, polyurethane or silicone groups.

The chemistry of epoxides has not been particularly studied in the manufacture of gels, much less so in the manufacture of hydrogels. However, epoxide chemistry involves reactions which are simple and quantitative at low temperatures. Moreover, it is not particularly sensitive to the presence of water, oxygen or impurities. Epoxides also generally have excellent mechanical and thermal properties. A large number of epoxides and amine are available; however, very few of them are water soluble.

WO2008079440A2 describes a method for preparing an epoxide-based superelastic hydrogel by reaction between polyetheramine and a polyglycidyl ether.

The publication by Paul Calvert, Prabir Patra, and Deepak Duggal “Epoxy hydrogels as sensors and actuators”, Proc. SPIE 6524, Electroactive Polymer Actuators and Devices (EAPAD) 2007, 65240M (4 Apr. 2007) describes hydrogels based on diepoxides and polyfunctional amines which are water soluble. This document describes the preparation of hydrogels from aliphatic polyamines and polyetheramines which have reacted with an aqueous solution of polyethylene glycol diglycidyl ether (PEGDGE).

In the current climate of the gradual reduction in fossil-based resources, it is becoming increasingly beneficial to replace products of fossil origin with other economically viable and environmentally acceptable products.

Moreover, one of the disadvantages of hydrogels is their poor mechanical properties. Several solutions can be envisaged to improve same. The proportion of rigid monomer or the degree of crosslinking can be increased. However, with a more dense network, the material becomes more brittle and has a reduced capacity for absorption.

It is therefore necessary to be able to provide hydrogels prepared from epoxide polymers of natural, non-fossil origin, having improved mechanical properties while being easy to obtain and retaining easy water absorption.

In the pursuit of their research using numerous studies, the Applicant company have found that a hydrogel based on polymers of natural, non-fossil origin, such as isosorbide epoxide polymers or monomers, had such characteristics.

Indeed, the rigid bicyclic structure of isosorbide epoxide makes it possible to improve the mechanical properties of the hydrogels prepared with these isosorbide epoxide polymers or monomers, while retaining very good water absorption characteristics by virtue of the hydrophilic nature of these compounds.

Other features and benefits of the present invention will become apparent on reading the following detailed description in conjunction with the appended FIGURE.

US2015/307650 A1 describes thermosetting and epoxide-based materials of plant origin.

US2008/009599 A1 describes thermoset epoxy polymers and more specifically thermoset polymers derived from renewable resources.

### SUMMARY OF THE INVENTION

A first subject matter of the present invention relates to a hydrogel based on water and on the reaction product of hydrosoluble monomeric or polymeric isosorbide epoxide and of hydrosoluble amine selected from a hydrosoluble diamine, triamine or polyamine.

A second subject matter of the present invention relates to a method for preparing a hydrogel according to the invention, comprising the following steps:

1) Mixing the hydrosoluble monomeric or polymeric isosorbide epoxide with a hydrosoluble amine,
2) Adding water to the previous mixture,
3) Mixing until a translucent liquid is obtained, and
4) Leaving to react.

A final subject matter of the present invention relates to the use of the hydrogel according to the invention in the medical, cosmetics, agricultural or optical fields, in the field of water treatment, hygiene products, in separation technology or in the energy sector.

### DETAILED DESCRIPTION

A hydrogel is proposed, based on water and on the reaction product of hydrosoluble monomeric or polymeric isosorbide epoxide and of hydrosoluble amine selected from a hydrosoluble diamine, triamine or polyamine.

“Hydrogel based on water” or “water-based hydrogel” is intended to mean a material consisting of a three-dimensional network obtained by crosslinking polymer chains, in which material water or an oil-in-water emulsion can be trapped. The crosslinked three-dimensional network, which is insoluble, is referred to hereinafter as hydrogel matrix.

According to the present invention, the hydrogel matrix is composed of monomeric or polymeric isosorbide epoxide having the following formula (I):

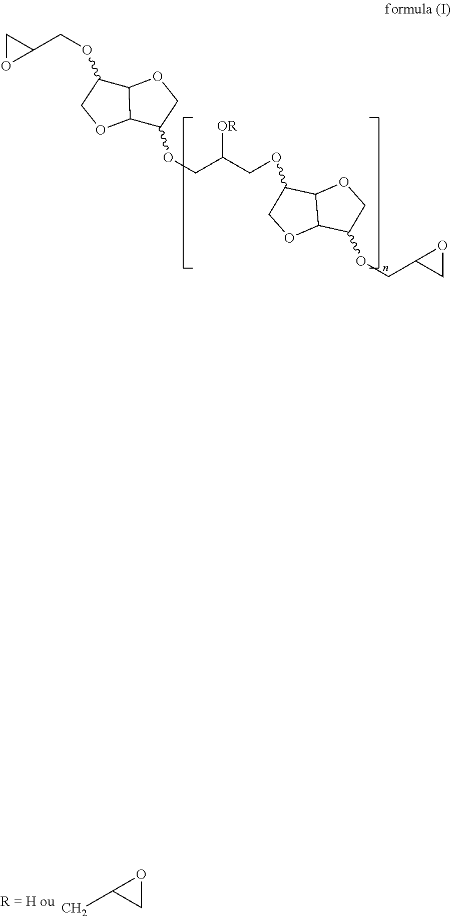

where n is an integer from 0 to 300, in particular from 0 to 10, and more particularly from 0 to 5.

The epoxide according to formula (I) may be manufactured according to the method described in application WO 2015/110758 A1.

It has the benefit of being bio-based and, unlike bisphenol A, is not an endocrine disruptor.

The isosorbide epoxide may be a mixture of different isosorbide epoxides which differ from one another by the substituent R and/or the subscript n.

The subscript n may range from 0 to 300, in particular be equal to 290, 280, 270, 260, 250, 240, 230, 220, 210, 200, 190, 180, 170, 160, 150, 140, 130, 120, 110, 100, 90, 80, 70, 60, 50, 40, 30, 20, 10, 9, 8, 7, 6, 5, 4, 3, 2, 1.

According to one embodiment, the subscript n may be between 0 and 290, 0 and 280, 0 and 270, 0 and 260, 0 and 250, 0 and 240, 0 and 230, 0 and 220, 0 and 210, 0 and 200, 0 and 190, 0 and 180, 0 and 170, 0 and 160, 0 and 150, 0 and 140, 0 and 130, 0 and 120, 0 and 110, 0 and 100, 0 and 90, 0 and 80, 0 and 70, 0 and 60, 0 and 50, 0 and 40, 0 and 30, 0 and 20, 0 and 10, 0 and 9, 0 and 8, 0 and 7, 0 and 6, 0 and 5.

According to one embodiment, the subscript n may be between 1 and 290, 1 and 280, 1 and 270, 1 and 260, 1 and 250, 1 and 240, 1 and 230, 1 and 220, 1 and 210, 1 and 200, 1 and 190, 1 and 180, 1 and 170, 1 and 160, 1 and 150, 1 and 140, 1 and 130, 1 and 120, 1 and 110, 1 and 100, 1 and 90, 1 and 80, 1 and 70, 1 and 60, 1 and 50, 1 and 40, 1 and 30, 1 and 20, 1 and 10, 1 and 9, 1 and 8, 1 and 7, 1 and 6, 1 and 5.

According to one embodiment, the subscript n may be between 2 and 290, 2 and 280, 2 and 270, 2 and 260, 2 and 250, 2 and 240, 2 and 230, 2 and 220, 2 and 210, 2 and 200, 2 and 190, 2 and 180, 2 and 170, 2 and 160, 2 and 150, 2 and 140, 2 and 130, 2 and 120, 2 and 110, 2 and 100, 2 and 90, 2 and 80, 2 and 70, 2 and 60, 2 and 50, 2 and 40, 2 and 30, 2 and 20, 2 and 10, 2 and 9, 2 and 8, 2 and 7, 2 and 6, 2 and 5.

According to one embodiment, the subscript n may be between 3 and 290, 3 and 280, 3 and 270, 3 and 260, 3 and 250, 3 and 240, 3 and 230, 3 and 220, 3 and 210, 3 and 200, 3 and 190, 3 and 180, 3 and 170, 3 and 160, 3 and 150, 3 and 140, 3 and 130, 3 and 120, 3 and 110, 3 and 100, 3 and 90, 3 and 80, 3 and 70, 3 and 60, 3 and 50, 3 and 40, 3 and 30, 3 and 20, 3 and 10, 3 and 9, 3 and 8, 3 and 7, 3 and 6, 3 and 5.

According to one embodiment, the subscript n may be between 4 and 290, 4 and 280, 4 and 270, 4 and 260, 4 and 250, 4 and 240, 4 and 230, 4 and 220, 4 and 210, 4 and 200, 4 and 190, 4 and 180, 4 and 170, 4 and 160, 4 and 150, 4 and 140, 4 and 130, 4 and 120, 4 and 110, 4 and 100, 4 and 90, 4 and 80, 4 and 70, 4 and 60, 4 and 50, 4 and 40, 4 and 30, 4 and 20, 4 and 10, 4 and 9, 4 and 8, 4 and 7, 4 and 6, 4 and 5.

According to one embodiment, the subscript n may be between 5 and 290, 5 and 280, 5 and 270, 5 and 260, 5 and 250, 5 and 240, 5 and 230, 5 and 220, 5 and 210, 5 and 200, 5 and 190, 5 and 180, 5 and 170, 5 and 160, 5 and 150, 5 and 140, 5 and 130, 5 and 120, 5 and 110, 5 and 100, 5 and 90, 5 and 80, 5 and 70, 5 and 60, 5 and 50, 5 and 40, 5 and 30, 5 and 20, 5 and 10, 5 and 9, 5 and 8, 5 and 7, 5 and 6.

According to one embodiment, the subscript n may be between 10 and 290, 10 and 280, 10 and 270, 10 and 260, 10 and 250, 10 and 240, 10 and 230, 10 and 220, 10 and 210, 10 and 200, 10 and 190, 10 and 180, 10 and 170, 10 and 160, 10 and 150, 10 and 140, 10 and 130, 10 and 120, 10 and 110, 10 and 100, 10 and 90, 10 and 80, 10 and 70, 10 and 60, 10 and 50, 10 and 40, 10 and 30, 10 and 20.

In the hydrogel, the monomeric or polymeric isosorbide epoxide of formula (I) is crosslinked using a crosslinking agent. The crosslinking agent is a hydrosoluble amine selected from a hydrosoluble diamine, triamine or polyamine. According to one embodiment, the hydrosoluble amine is selected from amino acids such as lysine, arginine, asparagine, glutamine, isophorone diamine, diaminodiphenylsulfone, hexamethylene diamine, m-xylenediamine and polyetheramines such as diaminopolypropylene glycol (Jeffamine D-230) and trimethylolpropane poly(oxypropylene)triamine (Jeffamine T-403) and mixtures thereof.

According to a preferred embodiment, the ratio of hydrosoluble monomeric or polymeric isosorbide epoxide equivalents to the number of N—H functions of the hydrosoluble amine is between 1:5 and 5:1, preferably between 1:2 and 2:1, and more preferentially 1:1. The optimum ratio is located between 2:1 and 1:2 with a maximum density for a ratio of 1:1.

“Water” is intended to mean demineralized water. According to a preferred embodiment, the hydrogel has a moisture content of 50 to 99%. The moisture content is measured using the TGA: TG209F1 iris apparatus from NETZSCH, according to the following method:

A few mg of product are deposited in an aluminum crucible. A heating ramp from 25° C. to 300° C. at 10° C./min under inert gas (nitrogen at a flow rate of 40 ml/min) is carried out.

When the hydrogel is in the presence of a stimulus to which it is sensitive, it swells or shrinks. As types of stimulus, mention may be made of pH, temperature, enzymes or other biochemical agents. It can also swell or shrink over time, based in particular on the environment in which it is located.

In summary, the hydrogel according to the invention has the following properties:
Bio-based
Adaptable, and
Controllable syneresis.

The mechanical properties and the crosslinking density of the hydrogel can be adjusted during the method for preparing the hydrogel.

Hydrogels can have different physical forms, for example:
a soft solid, such as a contact lens,
a compressed powder, such as used in the production of certain pills and capsules for the oral administration of active ingredients,
microparticles which may serve as vectors for medical applications, for example bioadhesive vectors during wound treatment, or else
a special coating for implants.
   According to a preferred embodiment, the hydrogel is biocompatible.

According to another preferred embodiment, the hydrogel further comprises an active ingredient. All the characteristics of the hydrogel described previously also apply to the hydrogel comprising an active ingredient.

“Active ingredient” is intended to mean any body, material or substance, whether pure or in a mixture, of chemical, biochemical or living nature, which has an effect or technical function in a field of industry, especially in the medical, cosmetics, agricultural or optical fields, in the field of water treatment, hygiene products, in separation technology or in the energy sector, etc. In the medical field, the active ingredient may be a pharmaceutical active substance which will be salted out under certain conditions. In the field of cosmetics, the active ingredient may be a skin-tightening molecule. In the optical field, the active ingredient may be a molecule salted out at the surface of the eye while contact lenses are being worn. In the field of water treatment, the active ingredient may be a decontaminant. In the field of hygiene products, the active ingredient may be a decontaminant. When the hydrogel is water-based, the active ingredient is dissolved in water. The solution is subsequently captured by the mesh of the network and is finally salted out by syneresis. When the hydrogel is based on an oil-in-water emulsion, the active ingredient is dissolved in the oily discontinuous phase of the emulsion. The emulsion is subsequently captured by the mesh of the network and is finally salted out by syneresis. The active ingredient is preferably a water-soluble active ingredient, preferably an odorizing molecule, a cosmetic active ingredient or a hydrosoluble pharmaceutical active ingredient. This active ingredient may for example be isosorbide, which is known for its wound-healing properties.

If the hydrogel does not comprise an active ingredient, it makes it possible to extract solid or particulate elements from a liquid medium containing this hydrogel. This hydrogel is of use in the medical, cosmetics, agricultural or optical fields, in the field of water treatment, hygiene products, in separation technology or in the energy sector. In the medical field, the hydrogel may extract toxic molecules present in the human body. In the field of cosmetics, the hydrogel may extract unwanted molecules present at the surface of the skin. In the field of water treatment, the hydrogel may extract active substances from waste water. In the field of hygiene products, the hydrogel may extract the surfaces of an unwanted molecule. In the field of separation technology, the hydrogel may extract the molecule from a solution which is to be purified.

“Extract solid or particulate elements from a liquid medium containing this hydrogel” is intended to mean the act of trapping, or capturing, elements present in a liquid medium then removing them from said medium.

According to another aspect of the present invention, the use of a hydrogel in the medical, cosmetics, agricultural or optical fields, in the field of water treatment, hygiene products, in separation technology or in the energy sector is proposed.

According to another aspect, a method for preparing a hydrogel according to the invention, comprising the following steps is proposed:

1) Mixing the hydrosoluble monomeric or polymeric isosorbide epoxide with a hydrosoluble amine,
2) Adding water to the previous mixture,
3) Mixing until a translucent liquid is obtained, and
4) Leaving to react at ambient temperature or at a temperature which may range up to 80° C.

According to a particular embodiment, the method for preparing a hydrogel according to the invention comprises the following steps:

1) Mixing the hydrosoluble monomeric or polymeric isosorbide epoxide with a hydrosoluble amine, preferably according to a ratio of epoxy functions to —NH functions of between 1:5 and 5:1, more preferentially between 1:2 and 2:1, and even more preferentially 1:1,
2) Adding water to the previous mixture,
3) Mixing until a translucent liquid is obtained,
4) Optionally pouring the translucent liquid obtained into a mold,
5) Leaving to react, preferably at ambient temperature or at a temperature which may range up to 80° C., and
6) Optionally removing the hydrogel thus formed from the mold.

According to another embodiment, the method for preparing a hydrogel according to the invention comprising the following steps:

1) Mixing the hydrosoluble monomeric or polymeric isosorbide epoxide with a hydrosoluble amine according to a ratio of epoxy functions to —NH functions of between 1:5 and 5:1, preferably between 1:2 and 2:1, and more preferentially 1:1,
2) Adding water to the previous mixture,
3) Mixing until a translucent liquid is obtained,
4) Pouring the translucent liquid obtained into a mold,
5) Leaving to react at ambient temperature or at a temperature which may range up to 80° C., and
6) Removing the hydrogel thus formed from the mold.

If an active ingredient is included in the hydrogel, then it is prepared as previously but with the addition of the active ingredient during step 2) described above.

According to another embodiment, steps 1) and 2) of the method for preparing the hydrogel according to the invention can be carried out successively in this order, or the other way round, or simultaneously.

### BRIEF DESCRIPTION OF THE DRAWINGS

Other features, details and advantages of the invention will appear from reading the following detailed description, and by analyzing the appended drawings, in which:

FIG. 1

FIG. 1 is a diagram showing the kinetics of the concentration of isosorbide salted out into water from the preparation of example 2.

### EXAMPLES

Reagents:

Isosorbide epoxide: Roquette.

### Lysine: Ajinomoto.

Isosorbide as active ingredient: Roquette.
Water: Demineralized water.

### Example 1: Preparation of a Hydrogel Containing 50% by Weight of Water

5 g of isosorbide epoxide (EEW=180 g/eq) (i.e. the product of formula (I) with R═H and n=0) are mixed with 1.03 g of lysine. 6.03 g of demineralized water are then added. Mixing is finally carried out in order to obtain a homogeneous preparation. The mixture is poured into a mold, then left to react at ambient temperature. The hydrogel is obtained after 72 h. The hydrogel thus obtained is removed from the mold.

### Example 2: Preparation of a Hydrogel Containing 50% by Weight of Water, Further Comprising Isosorbide as Active Ingredient

5 g of isosorbide epoxide (EEW=180 g/eq) are mixed with 1.03 g of lysine. 6.03 g of a 20% by weight aqueous solution of isosorbide are then added. Mixing is finally carried out in order to obtain a homogeneous preparation. The mixture is poured into a mold, then left to react at ambient temperature. The hydrogel is obtained after 72 h. The hydrogel containing the active ingredient thus obtained is removed from the mold.

### Example 3: Salting-Out Test of the Preparation of Example 2

The hydrogel containing the isosorbide obtained according to example 2 is subsequently submerged in a given volume of water. The content of isosorbide salted out is determined by gas chromatography in the form of trimethylsilyl derivatives and quantified by the internal calibration method as described below:

Name of apparatus: CPG, type Varian 3800 or Bruker 450 equipped with a split-splitless injector; a FID detector; a DB1 capillary column (J&W scientific ref 123-1033; 30 m in length; 0.32 mm in internal diameter; 1 micron film thickness.

Analysis conditions:

Column temperature: temperature program starting from injection, from 140° to 250° C. at a rate of 3° C./minute, then up to 300° C. at a rate of 10° C./min.
Injector temperature: 300° C.
Detector temperature: 300° C.

### Pressure: 10 psi

### Vector gas: Helium

Injection mode: split, with imperative “liner split”
Flow rate of split: 80 ml/minute
Hydrogen flow rate: 30 ml/minute
Air flow rate: 400 ml/minute
Volume injected: 1 microliter
Approximately precisely 1 g of product and 50 mg of internal standard (α-methyl-D-glucopyranoside) are weighed into a 100 ml beaker. Approximately 50 ml of pyridine (4-1) are added. This is left under magnetic stirring until complete dissolution is achieved.
1 ml of the solution, 1 ml of pyridine and 0.3 ml of BSTFA are deposited in a 2 ml dish with screw-on lid. The lid is closed. The container is agitated. It is left in a dry bath temperature controlled to 70° C. for 30 minutes, before injecting 1 microliter.

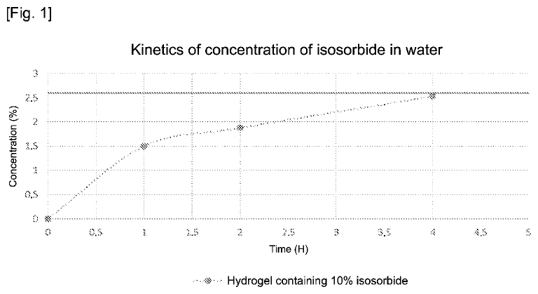

The kinetics of concentration of isosorbide salted-out into water is given in FIG. 1, for which the threshold of 2.5% corresponds to the maximum concentration which can be implemented in this test in light of the amount of isosorbide introduced and the known volume of water used for the salting out step.

This test shows that the hydrogel formed has a capacity for salting out isosorbide. The latter is completely salted out after 4 h.

As a result, the inventors have discovered that the hydrogel according to the invention makes it possible to salt out active ingredients, with this salting out time being adjustable based on the formulation of the hydrogel.

The hydrogel according to the invention, obtained according to a simple preparation method, is a good alternative to hydrogels based on polymers of fossil origin. Said hydrogel according to the invention has good water absorption, making it possible to advantageously salt out the active ingredient it contains. Due to these properties, the hydrogel according to the invention can therefore be used in various applications, particularly in the medical, cosmetics, agricultural or optical fields, in the field of water treatment, hygiene products, in separation technology or in the energy sector.

1-9. (canceled)

10. A hydrogel based on water and on the reaction product of hydrosoluble monomeric or polymeric isosorbide epoxide and of hydrosoluble amine selected from a hydrosoluble diamine, triamine or polyamine.

11. The hydrogel according to claim 10, wherein the hydrosoluble monomeric or polymeric isosorbide epoxide has the following formula (I): 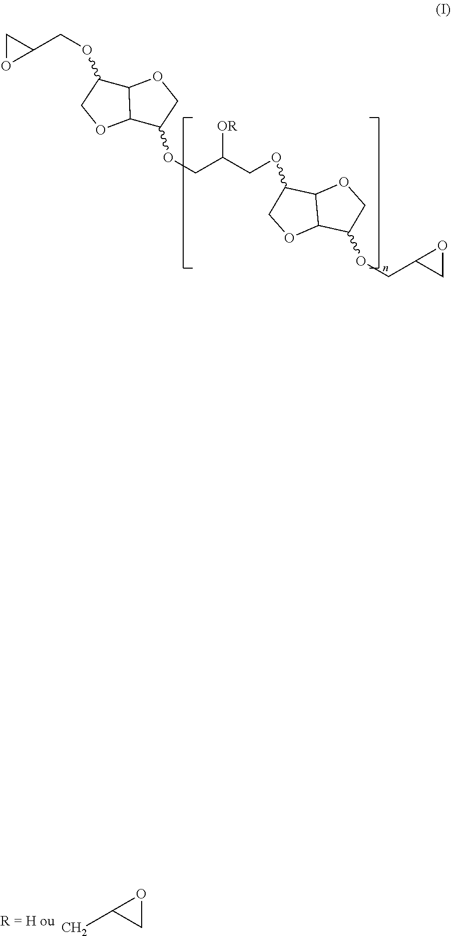
Where n is an integer from 0 to 300, preferably from 0 to 10, and more preferentially from 0 to 5.

12. The hydrogel according to claim 10, wherein the hydrosoluble diamine, triamine or polyamine is selected from lysine, arginine, asparagine, glutamine, isophorone diamine, diaminodiphenylsulfone, hexamethylene diamine, m-xylenediamine and polyetheramines such as Jeffamine D-230, Jeffamine T-403 and mixtures thereof.

13. The hydrogel according to claim 10, wherein the ratio of hydrosoluble monomeric or polymeric isosorbide epoxide equivalents to the number of N—H functions of the hydrosoluble amine is between 1:5 and 5:1, preferably between 1:2 and 2:1, and more preferentially 1:1.

14. The hydrogel according to claim 10, having a moisture content of 50 to 99%.

15. The hydrogel according to claim 10, further comprising a water-soluble active ingredient, preferably an odorizing molecule, a cosmetic active ingredient or a hydrosoluble pharmaceutical active ingredient and more preferentially isosorbide.

16. A method for preparing a hydrogel as defined in claim 10, comprising the following steps:
1) Mixing the hydrosoluble monomeric or polymeric isosorbide epoxide with a hydrosoluble amine,
2) Adding water to the previous mixture,
3) Mixing until a translucent liquid is obtained, and
4) Leaving to react.

17. The method for preparing a hydrogel according to claim 16, wherein step 2) comprises adding an active ingredient.

18. A use of a hydrogel as defined in claim 10 in the medical, cosmetics, agricultural or optical fields, in the field of water treatment, hygiene products, in separation technology or in the energy sector.
